(19) United States
(12) Patent Application Publication
(10) Pub. No.: US 2022/0296773 A1
(43) Pub. Date: Sep. 22, 2022

(54) USE OF IGSF10 IN PREPARATION OF BONE TISSUE REGENERATION PRODUCT

(71) Applicant: Shanghai Ninth People's Hospital, Shanghai Jiao Tong University School of Medicine, Shanghai (CN)

(72) Inventors: Xinquan JIANG, Shanghai (CN); Jin WEN, Shanghai (CN); Qianju WU, Shanghai (CN); Ran YAN, Shanghai (CN); Yuwei DENG, Shanghai (CN)

(73) Assignee: Shanghai Ninth People's Hospital, Shanghai JiaoTong University School of Medicine, Shanghai (CN)

(21) Appl. No.: 17/376,191

(22) Filed: Jul. 15, 2021

(30) Foreign Application Priority Data
Mar. 22, 2021 (CN) 2021103032733

(51) Int. Cl. A61L 27/22; A61K 38/18; A61K 39/395; A61L 27/12; A61L 27/10; A61L 27/26; A61L 27/54; A23L 33/17; A23L 33/00; A23K 20/147

(52) U.S. Cl. CPC A61L 27/227; A61K 38/1875; A61K 39/395; A61L 27/12; A61L 27/10; A61L 27/26; A61L 27/54; A23L 33/17; A23L 33/40; A23K 20/147; A61L 2430/34; A61L 2430/02; A61L 2300/256; A61L 2300/252; A23V 2002/00

(57) ABSTRACT

The present disclosure relates to the field of medicines, in particular to the use of IGSF10 in the preparation of a bone tissue regeneration product, especially the use of IGSF10 in combination with BMP in the preparation of a product for promoting bone tissue regeneration. The product includes a periodontal bone defect repair product, a jaw bone defect repair product and/or a skull defect repair product. The new use of IGSF10 of the present disclosure can reduce the effective concentration of BMP without obvious adverse reactions, thereby exploring a method to promote bone tissue regeneration and providing new ideas for the treatment of bone defects.

### CROSS REFERENCE TO RELATED APPLICATION

This application claims the benefits of priority to Chinese Patent Application No. CN 2021103032733, entitled “Use of IGSF10 in Preparation of Bone Tissue Regeneration Product”, filed with CNIPA on Mar. 22, 2021, the contents of which are incorporated herein by reference in its entirety.

### BACKGROUND

### Field of Disclosure

The present disclosure relates to the field of medicines, in particular, to the use of IGSF10 in the preparation of bone tissue regeneration products.

### Description of Related Arts

The regeneration of defective bones by tissue engineering is a hot research topic. Growth factors play an important role in regulating cell proliferation and migration, and extracellular matrix synthesis, which can effectively promote wound healing and promote the growth of bone tissue. Growth factors commonly used in clinic application include bone morphogenetic proteins (BMPs) that promote bone regeneration, platelet-derived growth factor (PDGF), enamel matrix protein (EMP), and amelogenin, et al. The BMP family are important growth factors known to promote the differentiation of pre-osteoblasts and pre-cementocytes. However, the above-mentioned factors are often accompanied with adverse reactions such as swelling and ectopic mineralization during clinical applications. Therefore, the finding of alternative or synergistic growth factors for realizing bone tissue repair has become a current research hotspot.

### SUMMARY

The present disclosure provides the use of IGSF10 in the preparation of bone tissue regeneration products, to solve the problems in the traditional technology.

The present disclosure provides the use of IGSF10 in the regeneration of bone tissues.

Preferably, the use is the use of IGSF10 in combination with one or more of bone morphogenetic protein (BMP), platelet-derived growth factor (PDGF), enamel matrix protein (EMP) and amelogenin in the regeneration of bone tissues.

The present disclosure further provides the use of IGSF10 in the preparation of bone tissue regeneration products.

Preferably, the use is the use of IGSF10 in combination with one or more of bone morphogenetic proteins (BMPs), platelet-derived growth factor (PDGF), enamel matrix protein (EMP) and amelogenin in the preparation of a product for promoting bone tissue regeneration.

The present disclosure further provides a product including a bone repair material and a growth factor loaded on the bone repair material, and the growth factor includes IGSF10.

Preferably, the growth factor in the product further includes one or more of BMPs, PDGF, EMP, and amelogenin.

The present disclosure further provides a method for repairing a bone defect, including administering the product to a patient with a bone defect.

As mentioned above, the use of IGSF10 of the present disclosure in the preparation of bone tissue regeneration products has the following beneficial effects: the effective concentration of BMP, PDGF, EMP or amelogenin could be reduced, and adverse reactions colud be decreased, such that a method to promote bone tissue regeneration is explored, and new ideas for the treatment of bone defects are provided.

### BRIEF DESCRIPTION OF THE DRAWINGS

FIG. 1 shows the osteogenic effects of IGSF10 and BMP2 on DPC and PDLC ((A): alizarin red staining; (B) calcium ion releasing from cells detection).

FIG. 2 shows the H&E histological examination of the repair effect of IGSF10 and BMP2 in a periodontal defect model.

FIG. 3 shows the Micro-CT result of IGSF10 and BMP2 in repairing rat periodontal bone defects.

FIG. 4 shows the Micro-CT effect of IGSF10 and BMP2 in repairing skull defects in vivo.

### DETAILED DESCRIPTION OF THE PREFERRED EMBODIMENTS

The present disclosure is the first to provide the use of IGSF10 in the regeneration of bone tissues.

Immunoglobulin superfamily member 10 (IGSF10) belongs to the immunoglobulin superfamily and act as a growth factor. Unless otherwise specified in the present disclosure, bone tissue regeneration includes cartilage tissue regeneration and bone tissue regeneration. Regeneration refers to the repair of damaged cells or tissues by the division and proliferation of neighboring healthy tissue cells.

Specifically, the use is the use of IGSF10 in the regeneration of bone tissues in mammals.

The mammals are preferably rodents, artiodactyls, perissodactyls, lagomorphs, primates, or the like. The primates are preferably monkeys, apes or humans.

In an embodiment, the applicaiton is the use of IGSF10 in promoting the regeneration of bone tissues. IGSF10 can promote bone tissue regeneration by inducing and maintaining the differentiation of bone or cartilage at an early stage of development.

The bone tissue may be, for example, periodontal bone, jaw bone, skull, tibia, fibula, femur and other bone tissues of the whole body skeletal system.

In a preferred embodiment, the use is the use of IGSF10 in periodontal bone defect repair, jaw bone defect repair and/or skull defect repair.

The IGSF10 may be a natural protein or a recombinant protein.

Further, the IGSF10 includes a protein formed by any IGSF10 sequence derived from human, murine, canine, bovine, porcine and the like. Information about the above-mentioned basic protein sequences from different sources may be retrieved from NCBI. In an embodiment, the IGSF10 is selected from human recombinant IGSF10, and the amino acid sequence is shown in SEQ ID NO. 1: SAFISPQGFMAPFGSLTLNMTDQSGNEANMVCSIQKPSRTSPIAFTEENDYIVLNTSFSTFLVCNIDYGHI QPVWQILALYSDSPLILERSHLLSETPQ (SEQ ID NO.1)

In a preferred embodiment, the use is the use of IGSF10 in combination with one or more of bone morphogenetic protein (BMP), platelet-derived growth factor (PDGF), enamel matrix protein (EMP), and amelogenin in the regeneration of bone tissues. The combination application can reduce the effective concentration of several other growth factors, thereby reducing adverse reactions.

In an embodiment, the BMP is BMP-2.

In an embodiment, the concentration of each growth factor may be 200-600 ng/mL when used in combination. For example, the concentration may be selected from one of the following concentration ranges: 200-250 ng/mL, 250-300 ng/mL, 300-350 ng/mL, 350-400 ng/mL, 400-450 ng/mL, 450-500 ng/mL, 500-550 ng/mL, and 550-600 ng/mL.

Those skilled in this field understand that the buffer for diluting the growth factors of IGSF10, BMPs, PDGF, EMP or amelogenin may be commonly used buffer such as normal saline, PBS, or Tris, provided that the nature of the growth factors is not changed.

The present disclosure further provides the use of IGSF10 in the preparation of bone tissue regeneration products.

Specifically, the use is the use of IGSF10 in the preparation of mammalian bone tissue regeneration products.

The mammals are preferably rodents, artiodactyls, perissodactyls, lagomorphs, primates, or the like. The primates are preferably monkeys, apes, or humans.

In an embodiment, the use is the use of IGSF10 in the preparation of a product for promoting bone tissue regeneration.

The bone tissue may be, for example, periodontal bone, jaw bone, or skull.

In a preferred embodiment, the use is the use of IGSF10 in the preparation of a product for repairing the periodontal bone defect, jaw bone defect and/or skull defect. In an embodiment, the use is the use of IGSF10 in the preparation of a product for inducing and maintaining the differentiation of bone or cartilage at an early stage of development.

The IGSF10 may be a natural protein or a recombinant protein.

Further, the IGSF10 includes a protein formed by any IGSF10 sequence derived from human, murine, canine, bovine, porcine and the like. Information about the above-mentioned basic protein sequences from different sources may be retrieved from NCBI. In an embodiment, the IGSF10 is selected from human recombinant IGSF10, and the amino acid sequence is shown in SEQ ID NO. 1: SAFISPQGFMAPFGSLTLNMTDQSGNEANMVCSIQKPSRTSPIAFTEENDYIVLNTSFSTFLVCNIDYGHI QPVWQILALYSDSPLILERSHLLSETPQ (SEQ ID NO.1)

In an embodiment, in addition to IGSF10, the bone tissue regeneration product further includes one or more of bone morphogenetic proteins (BMPs), platelet-derived growth factor (PDGF), and enamel matrix protein (EMP). That is, in an embodiment, the use is the use of IGSF10 in combination with one or more of BMPs, PDGF, EMP and amelogenin in the preparation of a product for promoting bone tissue regeneration. The combination of the above growth factors can reduce the effective concentration of several other growth factors, thereby reducing adverse reactions.

In an embodiment, the concentration of IGSF10 and/or other growth factors may be 200-600 ng/mL when the product is used. For example, the concentration may be selected from one of the following concentration ranges: 200-250 ng/mL, 250-300 ng/mL, 300-350 ng/mL, 350-400 ng/mL, 400-450 ng/mL, 450-500 ng/mL, 500-550 ng/mL, and 550-600 ng/mL.

Those skilled in this field understand that the buffer for diluting IGSF10, BMPs, PDGF, EMP or amelogenin may be a commonly used buffer such as normal saline, PBS, or Tris, provided that the nature of the growth factors is not changed.

The products include, but are not limited to, drugs, health care products, foods, and consumables. IGSF10 is the only effective component or one of the effective components of the product.

The product may be a single-component substance or a multi-component substance.

In an embodiment, the product is a drug, and the drug further includes a pharmaceutically acceptable excipient.

“Pharmaceutically acceptable” means that when the molecular entities and compositions are properly administered to animals or humans, they will not produce adverse, allergic, or other untoward reactions.

Furthermore, the pharmaceutically acceptable excipients should be compatible with the effective components, that is, able to blend with the effective components without greatly reducing the effect of the drug under normal circumstances. Specific examples of substances that may serve as pharmaceutically acceptable carriers or excipients include saccharides (such as lactose, glucose, and sucrose), starches (such as corn starch and potato starch), cellulose and derivatives thereof (such as sodium methyl cellulose, ethyl cellulose and methyl cellulose), tragacanth powder, malt, gelatin, talc, solid lubricants (such as stearic acid and magnesium stearate), calcium sulfate, vegetable oils (such as peanut oil, cottonseed oil, sesame oil, olive oil, corn oil and cocoa butter), polyols (such as propylene glycol, glycerol, sorbitol, mannitol and polyethylene glycol), alginic acids, emulsifiers (such as Tween), wetting agents (such as sodium lauryl sulfate), coloring agents, flavoring agents, tableting agents, stabilizers, antioxidants, preservatives, pyrogen-free water, isotonic saline solution, and phosphate buffer. The above substances are used as needed, to increase the stability of the formulation, help improve the activity or bio-availability of the formulation, or produce an acceptable mouthfeel or odor in the case of oral administration.

In another embodiment, the product is a consumable.

Specifically, the consumable may be constructed by loading IGSF10 alone or in combination with other growth factors onto a certain carrier. For example, IGSF10 and/or one or more of BMPs, PDGF, EMP and amelogenin are loaded onto the bone repair material to form the consumable. The bone repair material, as a bridge connecting seed cells and regenerated tissues, has a structure and function similar to those of natural bones and contains material with osteoinductive activity. The bone repair material may be a hydroxyapatite scaffold, a calcium phosphate scaffold, or a bioglass scaffold.

The consumables may be prepared by loading growth factors onto the bone repair material in advance. Or, the consumables may be prepared on the spot, that is, the growth factors and the bone repair material are stored separately, and then the bone repair material is loaded with the growth factors during when application is started. The loading method is: slowly adding the growth factors onto the bone repair material dropwise, transferring the bone repair material added with the growth factors to a constant temperature incubator, standing for 1.5 to 2.5 hours before use. The temperature of the constant temperature incubator is preferably 25-37° C.

In an embodiment, the growth factors include IGSF10 and BMP2, which are loaded on the bone repair material in a 1:1 ratio. Of course, the ratio between the growth factors may be adjusted according to specific experimental conditions.

The present disclosure further provides a bone tissue regeneration product, including a bone repair material and growth factors loaded onto the bone repair material, and the growth factor includes IGSF10.

In an embodiment, the growth factors in the product further include one or more of BMPs, PDGF,

EMP and amelogenin.

In an embodiment, the bone repair material is a hydroxyapatite scaffold.

The present disclosure provides a method for preparing a bone tissue regeneration product, including adding growth factors to a bone repair material, allowing the bone repair material added with the growth factors to stand for 1.5-2.5 hours at a constant temperature; the growth factors include IGSF10.

Specifically, the preparation method is as follows: slowly adding the growth factor onto the bone repair material dropwise, transferring the bone repair material added with the growth factor to a thermostatic incubator, and standing for 1.5 to 2.5 hours before use. The temperature of the thermostatic incubator is preferably 25-37° C.

The growth factors further include one or more of BMP, PDGF, EMP and amelogenin;

The ratio between the growth factors is adjusted according to the actual experiments.

The ratio of the total mass of the growth factors to the total mass volume of the bone repair material is 0.1:1-15:1, and the unit is ng: mm³. For example, the ratio may be 0.1:1˜1:1, 1:1˜2:1, 2:1˜5:1, 5:1˜8:1, 8:1˜11:1 or 11:1˜15:1.

The present disclosure further provides a method for repairing a bone defect, including the following operations: administering the bone tissue regeneration product to a patient with a bone defect.

Specifically, the bone tissue regeneration product is administered to a bone defect site of the patient with the bone defect.

The amount of growth factors in the product meets the requirement of a therapeutically effective amount. The “therapeutically effective amount” refers to the amount of growth factors that is effective in treating bone defects, especially periodontal bone defects, jaw bone defects, and/or skull defects.

In an embodiment, the therapeutically effective amount is 1-160 ng. For example, the therapeutically effective amount may be a range, and the range is one of 1-10 ng, 10-20 ng, 20-70 ng, 70-120 ng, and 120-160 ng.

The bone defect refers to a bone shortage caused by various reasons, such as trauma or surgery. Bone defects often result in bone nonunion, delayed union or nonunion, and local dysfunction.

The repairing method further includes a series of routine operations, such as anesthesia, incision of the skin, suture, and stitch removal.

The repairing method further includes treating the defect site with a substance before administering the bone tissue regeneration product.

The repairing method may be used in humans or other mammals.

The embodiments of the present disclosure will be described below through exemplary embodiments. Those skilled in this field can easily understand other advantages and effects of the present disclosure according to contents disclosed by the specification. The present disclosure may also be implemented or applied through other different specific implementation modes. Various modifications or changes may be made to all details in the specification based on different points of view and applications without departing from the spirit of the present disclosure.

Before further describing the specific embodiments of the present disclosure, it is understood that the scope of the present disclosure is not limited to the specific embodiments described below; It is also needed to be understood that the terminology of the disclosure is used to describe the specific embodiments, and not to limit the scope of the disclosure; In the present specification and claims, the singular forms “a”, “an” and “the” include the plural forms, unless specifically stated otherwise.

When the numerical values are given by the embodiments, it is needed to be understood that the two endpoints of each numerical range and any value between the two endpoints may be selected unless otherwise stated. Unless otherwise defined, all technical and scientific terms used in the present disclosure have the same meaning as commonly understood by one skilled in the field. In addition to the specific method, equipment and material used in the embodiments, any method, equipment and material in the existing technology similar or equivalent to the method, equipment and material mentioned in the embodiments of the present disclosure may be used to realize the invention according to the understanding of the existing technology by those skilled in the art, and the present disclosure.

The recombinant IGSF10 protein used in the embodiments of the present disclosure is purchased from Abcam, and the article number is ab166199.

### Embodiment 1 Alkaline Phosphatase Staining and Calcium Releasing Detection

1.1 DPCs and PDLCs are inoculated in a 24-well plate ata density of 5×10⁴/mL and coculture with different concentration of IGSF10 or BMP-2 under osteogenic medium. After 9 d of culture, alizarin red staining is performed to observe the calcium deposition.

1.2 The amount of cells required for each assay (2×10⁵ cells) is obtained. The cells are washed with PBS, resuspended in 500 μL of calcium ion detection buffer and placed on ice. The cells are homogenized by pipetting up and down for several times or using a homogenizer. The samples are centrifuged at 4° C. at the highest speed for 2-5 min to remove any insoluble material. The supernatant is collected and transferred to a filter. Two duplicative samples are prepared. All reagents are restored to room temperature. Reaction wells are set up: standard wells=50 μL standard diluent; Sample wells=1-50 pL sample (the volume is adjusted to 50 μL/well with dH2O). Adding 90 μL of color-developing agent to each well containing standards, samples or controls. Adding 60 μL of calcium detection buffer to each well. Mixing and incubating at room temperature for 5-10 minutes, avoiding light. Measuring with a microplate reader (OD575 nm).

As shown in FIG. 1, alizarin red ARS staining shows that IGSF10 has a strong ability to promote the formation of calcium nodule when the inducing osteogenic solution is present. At the same time, the mineralization promotion effects of IGSF10 and BMP2 are compared, and the results show that IGSF10 is more effective than BMP2 in vitro. However, the quantitative results show no statistical difference.

### Embodiment 2 Periodontal Defect Experiment in Rats

The surgical region is cleaned and disinfected with 75% ethanol, and all the surgical instruments are thoroughly cleaned and disinfected to minimize contamination. Rats are anesthetized by inhaling a combination of 4% (wt/vol) isoflurane and oxygen. The necessary dose of lidocaine is calculated based on 5 mg/kg after weighing the body and injected subcutaneously on the back. After anesthetized, the supply amount of the nozzle of isoflurane is adjusted to 2.5% (wt/vol) for maintenance. To prevent dryness, eye ointment (lubricant) is applied to the eyes of the rats. After the skin preparation on the surgical region, using povidone-iodine topical antiseptic and sterile saline alternately to scrub outward and spirally to disinfect the skin.

At the inferior margin of the mandible, the skin near the masseter is incised and extended backwards. After the muscle is incised and separated, regions of the mandible and the first molar can be seen. After the distal roots of the first molar as well as the first molar are exposed, a 3×2×1 mm defect is prepared using a No. 4 round drill. Buccal roots and the cementum are removed. IGSF10 and BMP2 are loaded with the same dose onto hydroxyapatite scaffolds. The muscle is repositioned with absorbable nylon, the wound is sutured, and the skin is cleaned.

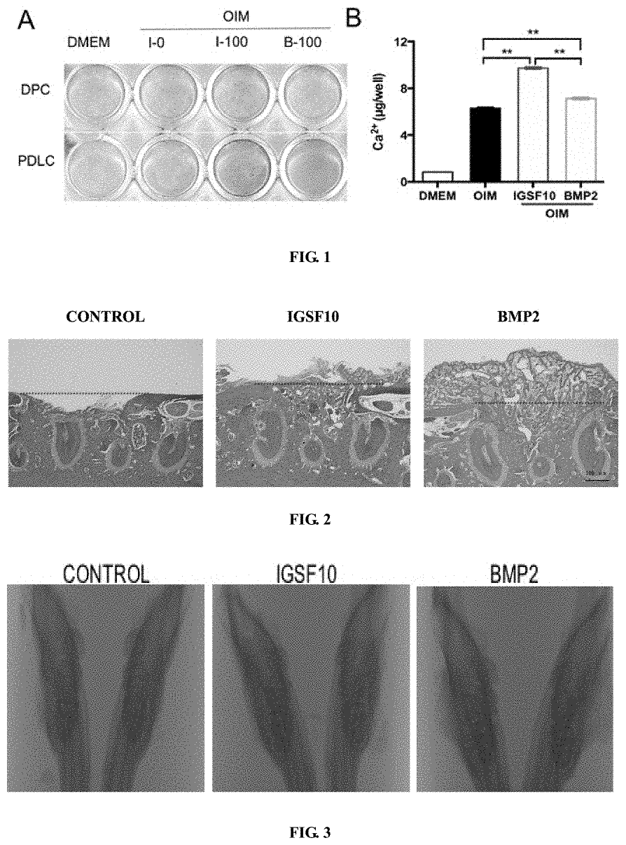

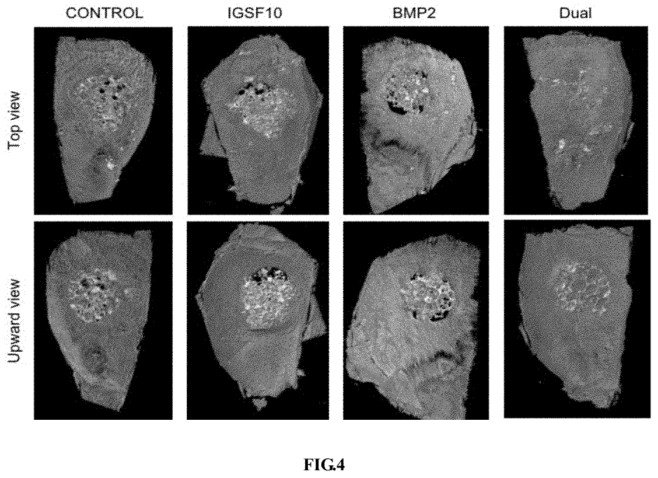

As shown in FIGS. 2-3, IGSF10 significantly promotes the regeneration of bone tissue at the defect site, with regular morphology. In the BMP2 group, the stimulation is stronger, with the bone tissue showing expansive growth and the morphology being irregular. Something similar to heterotopic mineralization occurs.

### Embodiment 3 Skull Defect Experiment in Mice

Male C57/BL6 mice are selected and weighed, and then anesthetized with isoflurane (5% for anesthetic induction and 1-3% for anesthesia maintenance). The anesthetic status is monitored by pinching the toes of the hind limbs. The respiratory rates are observed at least every 5 minutes. The mice are placed in a prone position, and the surgical plate is kept warm by a circulating warm water blanket. After shaving and disinfecting the skin, the skulls of the mice are disinfected with 70% alcohol. The surgical operators wear clean laboratory gowns, head caps, face masks and sterile gloves. 0.2 mL lidocaine is injected into the incision site under local anesthesia. A 2-cm-long incision is made with a blade along the midline of the skull. The skin and periosteum are bluntly dissected. A round bone defect is prepared on each side of the midline of the skull by using a trephine (outer diameter: 4 mm) under the condition of saline cooling.

In the in-vivo skull defect experiment, IGSF10 and BMP2 with the same dose are loaded onto the hydroxyapatite scaffold. The specific operation method is as follows: according to a ratio 1.3:1 (ng: mm³) of the total mass of IGSF10 and BMP2 to the volume of the hydroxyapatite scaffold, respectively. Moreover, for the combination application, the IGSF10 and BMP2 (R&D, USA) are loaded dropwise onto the hydroxyapatite scaffold at a ratio of 1:1 using a pipettor. The hydroxyapatite scaffold loaded with IGSF10 and BMP2 are transferred to a conventional incubator and allowed to stand for 2 hours, and the solution is loaded into the hydroxyapatite scaffold through the siphoning effect of the porous structure of the hydroxyapatite scaffold for subsequent in vivo experiments. After putting differently processed materials into the skull defect, the wounds are sutured with 5-0 non-absorbable suture materials. The sutures are removed 10-14 days after surgery. All procedures are carried out under aseptic conditions. 8 weeks after surgery, samples are collected and fixed with 4% paraformaldehyde for 2 days, then micro-CT detection is performed.

As shown in FIG. 4, IGSF10 and BMP2 with the same dose are loaded on the hydroxyapatite scaffold, both of them can effectively stimulate the formation of new bone. The results of the combined loading show that the skull defect site is completely replaced by new bone, suggesting that IGSF10 greatly improves the osteogenic effect of BMP2.

The above results show that the use of IGSF10 for bone tissue regeneration and repair can synergize or replace BMP currently in routine clinical use and thereby reducing the dosage of BMP and avoiding adverse reactions. Therefore, promoting the clinical application of IGSF10 is feasible.

The above embodiments are intended to illustrate the disclosed embodiments of the present disclosure and are not understood as restrictions on the present disclosure. In addition, various modifications of the present disclosure, as well as variations of the methods of the present disclosure, will be apparent to those skilled in the art without departing from the scope of the present disclosure. While the disclosure has been described in detail in connection with various specific preferred embodiments thereof, however, it should be understood that the present disclosure should not be limited to these specific embodiments. In fact, various modifications to the present disclosure as apparent to those skilled in the art are intended to be included within the scope of the present disclosure.

What is claimed is:

1. The use of IGSF10 in the preparation of a bone tissue regeneration product.

2. The use according to claim 1, wherein the use is a use of IGSF10 in the preparation of a mammalian bone tissue regeneration product.

3. The use according to claim 1, wherein the use is a use of IGSF10 in combination with one or more growth factors of BMP, PDGF, EMP, and amelogenin in the preparation of a product for promoting bone tissue regeneration.

4. The use according to claim 3, wherein the IGSF10, BMP, PDGF, EMP or amelogenin is selected from a natural protein or a recombinant protein.

5. The use according to claim 1, wherein the product comprises a periodontal bone defect repair product, a jaw bone defect repair product, and/or a skull defect repair product.

6. The use according to claim 1, wherein the product is drug, health care product, food, or consumable.

7. A bone tissue regeneration product, comprising a bone repair material and a growth factor loaded onto the bone repair material, wherein the growth factor includes IGSF10.

8. The bone tissue regeneration product according to claim 7, wherein the growth factor in the product further includes one or more of BMP, PDGF, EMP, and amelogenin.

9. The bone tissue regeneration product according to claim 7, wherein the bone repair material is a hydroxyapatite scaffold, a calcium phosphate scaffold, or a bioglass scaffold.

10. A method for preparing a bone tissue regeneration product, comprising: adding a growth factor to a bone repair material; and allowing the bone repair material added with the growth factor to stand for 1.5-2.5 hours ata constant temperature, wherein the growth factor includes IGSF10.

11. The method according to claim 10, further comprising one or more of the following:
1) the growth factor further includes one or more of BMP, PDGF, EMP and amelogenin;
2) the constant temperature is 25-37° C.;
3) the ratio of the total mass of the growth factor to the total mass of the bone repair material is 0.1:1˜15:1.

12. A method for repairing a bone defect, comprising the following operations: administering the bone tissue regeneration product according to claim 7 to a bone defect site.

13. The method for repairing a bone defect according to claim 12, comprising: administering the bone tissue regeneration product containing an effective amount of growth factor according to claim 7 to a bone defect site.

14. The method for repairing a bone defect according to claim 12, wherein the method can be used to repair a bone defect in mammals.
